(19) United States
(12) Patent Application Publication
(10) Pub. No.: US 2026/0240427 A1
(43) Pub. Date: Aug. 20, 2026

(54) METHOD AND SYSTEM FOR INTELLIGENT SEGMENTATION OF FUNDUS BLOOD VESSELS BASED ON RETINAL ANGIOGRAPHY

(71) Applicant: The Eye Hospital of Wenzhou Medical University, Wenzhou (CN)

(72) Inventors: Zhong Lin, Wenzhou (CN); Lei Wang, Wenzhou (CN); Jue Lin, Wenzhou (CN); Wei Lin, Wenzhou (CN); Riyan Zhang, Wenzhou (CN)

(21) Appl. No.: 19/078,031

(22) Filed: Mar. 12, 2025

(30) Foreign Application Priority Data
Feb. 19, 2025 (CN) 202510185986.2

(51) Int. Cl. A61B 3/00; A61B 3/12; G06V 10/26; G06V 10/30; G06V 10/34; G06V 10/764; G06V 10/774; G06V 10/776; G06V 10/80; G06V 10/82; G06V 20/70

(52) U.S. Cl. CPC A61B 3/0025; A61B 3/1241; G06V 10/26; G06V 10/30; G06V 10/34; G06V 10/764; G06V 10/774; G06V 10/776; G06V 10/806; G06V 10/82; G06V 20/70; G06V 2201/03

(57) ABSTRACT

Provided is a method and system for intelligent segmentation of fundus blood vessels based on retinal angiography. The method includes: acquiring fundus images with retinal angiography technology; performing a preprocessing operation on the fundus images to obtain preprocessed images; performing a superpixel segmentation on the preprocessed images to obtain segmented areas; performing a fundus blood vessel recognition on the segmented areas to obtain a recognition result; and constructing a trained fundus blood vessel segmentation model, and performing a feature segmentation on the recognition result by using the fundus blood vessel segmentation model to obtain a segmentation result. This method performs a multi-level feature extraction and fusion through the intelligent segmentation model, realizes effective extraction of multi-dimensional features of the fundus images, such as color, texture, shape and intensity, and improves segmentation accuracy through the feature fusion.

### CROSS-REFERENCE TO RELATED APPLICATION

This application claims priority of Chinese Patent Application No. 202510185986.2, filed on Feb. 19, 2025, the entire contents of which are incorporated herein by reference.

### TECHNICAL FIELD

The present disclosure relates to the technical field of medical image processing, and in particular, to a method and system for intelligent segmentation of fundus blood vessels based on retinal angiography.

### BACKGROUND

The detection and analysis of fundus blood vessels are of great significance in clinical medicine, especially in the diagnosis of ophthalmology and related diseases such as diabetes and cardiovascular diseases. Currently, technologies such as retinal angiography (FFA) and optical coherence tomography (OCT) are widely used to obtain fundus blood vessel images. Retinal angiography can provide clear images of fundus blood vessels. By analyzing these images, information about arteries and veins as well as early warning of potential diseases can be obtained. However, due to the complexity of fundus images, there are still many challenges in automating this process.

First, existing fundus blood vessel segmentation methods often rely on manual feature extraction, resulting in insufficient segmentation accuracy, inability to fully capture complex structures, and poor generalization ability of segmentation results. Secondly, many traditional algorithms have low execution efficiency and cannot meet the needs of real-time processing. Especially when processing full field of view images, the bottleneck of processing speed significantly affects the feasibility of clinical applications. In addition, fundus images are often affected by noise and artifacts. The existing technology is not capable of noise removal and artifact processing, which reduces the reliability of blood vessel segmentation. Existing technologies still lack intelligent analysis capabilities. Many methods only focus on image segmentation itself and do not conduct comprehensive analysis of blood vessel features (such as arteriovenous classification and blood vessel diameter calculation), which limits their ability to provide more comprehensive physiological and pathological information. Finally, many segmentation models are trained on specific datasets and lack the ability to adapt to different patients and different types of images, resulting in poor performance on new data. Therefore, it is very necessary to design a method and system for intelligent segmentation of fundus blood vessels based on retinal angiography.

### SUMMARY

An objective of the present disclosure is to provide a method and system for intelligent segmentation of fundus blood vessels based on retinal angiography to perform a multi-level feature extraction and fusion through an intelligent segmentation model, realize effective extraction of multi-dimensional features of fundus images, such as color, texture, shape and intensity, and improve segmentation accuracy through the feature fusion.

To achieve the above objective, the present disclosure provides solutions as follows.

A method for intelligent segmentation of fundus blood vessels based on retinal angiography is provided, which includes the following steps:
acquiring fundus images by a retinal angiography technology, where the fundus images include: a full field of view image and a local enhanced image;
performing a preprocessing operation on the fundus images to obtain preprocessed images;
performing a superpixel segmentation on the preprocessed images to obtain segmented areas;
performing a fundus blood vessel recognition on the segmented areas to obtain a recognition result, where the recognition result includes: an optic disc diameter, an arteriovenous blood vessel classification result, and an arteriovenous ratio; and
constructing a trained fundus blood vessel segmentation model, and performing a feature segmentation on the recognition result by using the fundus blood vessel segmentation model to obtain a segmentation result, where a training process of the fundus blood vessel segmentation model includes: a feature extraction, a feature integration and a loss function optimization.

Optionally, the full field of view image is an image covering an entire fundus range; the size of the local enhanced image is 1024*1024 pixels, and the number of blood vessels in the local enhanced image is greater than 20.

Optionally, performing the preprocessing operation on the fundus images to obtain the preprocessed images includes:
performing a denoising processing on the fundus images by using a Gaussian filter to obtain denoised images;
performing a histogram equalization processing on the denoised images to obtain enhanced images; and
performing a binarization processing on the enhanced images to obtain the preprocessed images.

Optionally, performing the superpixel segmentation on the preprocessed images to obtain the segmented areas includes:
determining the number of superpixels according to the size of the preprocessed image;
obtaining a superpixel size according to the number of superpixels, and determining a seed point according to the superpixel size;
extracting seed vectors of the seed point, and performing superpixel allocation according to a distance between the seed vectors to obtain an allocation result; and
performing a superpixel updating according to the allocation result to obtain the segmented areas.

Optionally, the distance between the seed vectors is calculated as follows:

$$Dis = \sqrt{\begin{matrix}{( {x}_{p} - {x}_{{c}_{k}} )}^{2} + {( {y}_{p} - {y}_{{c}_{k}} )}^{2} + {( \frac{{L}_{p} - {L}_{{c}_{k}}}{{D}_{L}} )}^{2} + \\ {( \frac{{a}_{p} - {a}_{{c}_{k}}}{{D}_{a}} )}^{2} + {( \frac{{b}_{p} - {b}_{{c}_{k}}}{{D}_{b}} )}^{2}\end{matrix}} ;$$

where: Dis is the vector distance, (xₚ,yₚ) is the position of pixel p, (x_{c}_{ₖ},y_{c}_{ₖ}) is the position of seed point cₖ, Lₚ, aₚ, and bₚ are different color values of the pixels in color space, L_{c}_{ₖ}, a_{c}_{ₖ}, and b_{c}_{ₖ} are different color values of the seed point in the color space, D_{L}, Dₐ, and D_{b} are color coordinate weights of different colors.

Optionally, performing the fundus blood vessel recognition on the segmented areas to obtain the recognition result includes:
performing a regional feature extraction on the segmented areas, and selecting an optic disc area according to regional features;
obtaining an optic disc edge of the optic disc area by a threshold segmentation method, and obtaining the optic disc diameter according to the optic disc edge;
performing a blood vessel classification on the segmented areas according to blood vessel colors to obtain the arteriovenous blood vessel classification result; and
performing a skeletonization processing and a window sliding on the blood vessels to obtain arteriovenous blood vessel diameters, and obtaining the arteriovenous ratio through the arteriovenous blood vessel diameters.

Optionally, the training process of the fundus blood vessel segmentation model includes:
inputting historical fundus images as a training set into an initial model to perform the feature extraction to obtain recognition features, where the recognition features include: color features, texture features, edge features, shape features, and intensity features;
performing a feature fusion on the recognition features to obtain fused features, and performing a standardization processing on the fused features to obtain integrated features;
performing a loss optimization on the integrated features through a loss function to obtain training output; and
performing a manual annotation on the historical fundus images to obtain annotated images, and using the annotated images as a verification set to verify the training output to obtain the fundus blood vessel segmentation model.

Optionally, the initial model includes a multi-scale convolution layer, a hole convolution layer and a feature reconstruction layer connected in sequence; the multi-scale convolution layer is composed of multiple convolution layers, and the different convolution layers have built-in convolution kernels of different sizes; and the feature reconstruction layer has a built-in deconvolution algorithm.

Optionally, an expression of the loss function is:

$$L = - \frac{1}{N} {\sum }_{i = 1}^{N} ( ω \cdot {y}_{i} log ( {p}_{i} ) + ( 1 - ω ) \cdot ( 1 - {y}_{i} ) log ( 1 - {p}_{i} ) ) + λ \cdot ( 1 - \frac{2 \vert y ⋂ p \vert}{\vert y \vert + \vert p \vert} ) ;$$

where: L is the loss function, N is the total number of samples, y is a true label, p is a prediction probability, ω is a sample weight, λ is a coefficient weight, and i is the i-th pixel.

A system for intelligent segmentation of fundus blood vessels based on retinal angiography is provided, which includes:
an image acquisition module configured to acquire fundus images by a retinal angiography technology;
an image processing module configured to perform a preprocessing operation on the fundus images to obtain preprocessed images;
an image segmentation module configured to perform a superpixel segmentation on the preprocessed images to obtain segmented areas;
an image recognition module configured to perform a fundus blood vessel recognition on the segmented areas to obtain a recognition result; and
a model construction module configured to construct a trained fundus blood vessel segmentation model, and perform a feature segmentation on the recognition result by using the fundus blood vessel segmentation model to obtain a segmentation result.

According to specific embodiments provided by the present disclosure, the following technical effects are disclosed: the present disclosure provides the method for intelligent segmentation of fundus blood vessels based on retinal angiography, the method including: acquiring fundus images by a retinal angiography technology; performing a preprocessing operation on the fundus images to obtain preprocessed images; performing a superpixel segmentation on the preprocessed images to obtain segmented areas; performing a fundus blood vessel recognition on the segmented areas to obtain a recognition result; and constructing a trained fundus blood vessel segmentation model, and performing a feature segmentation on the recognition result by using the fundus blood vessel segmentation model to obtain a segmentation result.

### BRIEF DESCRIPTION OF THE DRAWINGS

In order to more clearly illustrate the embodiments of the present disclosure or the technical solutions in the existing technology, the accompanying drawings required for use in the embodiments will be briefly introduced below. Obviously, the accompanying drawings described below are only some embodiments of the present disclosure. For those of ordinary skill in the art, other drawings can be obtained based on these drawings without paying creative labor.

FIG. 1 is a flow chart of a method for intelligent segmentation of fundus blood vessels according to an embodiment of the present disclosure;

FIG. 2 is a flow chart of a preprocessing operation according to an embodiment of the present disclosure;

FIG. 3 is a flow chart of superpixel segmentation according to an embodiment of the present disclosure;

FIG. 4 is a flow chart of fundus blood vessel recognition of according to an embodiment of the present disclosure; and

FIG. 5 is a flow chart of training of a fundus blood vessel segmentation model according to an embodiment of the present disclosure.

### DETAILED DESCRIPTION

The technical solutions in the embodiments of the present disclosure will be described clearly and completely below in conjunction with the accompanying drawings in the embodiments of the present disclosure. Obviously, the described embodiments are only part of the embodiments of the present disclosure, rather than all the embodiments. Based on the embodiments of the present disclosure, all other embodiments obtained by those of ordinary skill in the art without making any creative work shall fall within the scope of protection of the present disclosure.

In order to make the above-mentioned objects, features and advantages of the present disclosure more obvious and understandable, the present disclosure is further described in detail below with reference to the accompanying drawings and specific implementations.

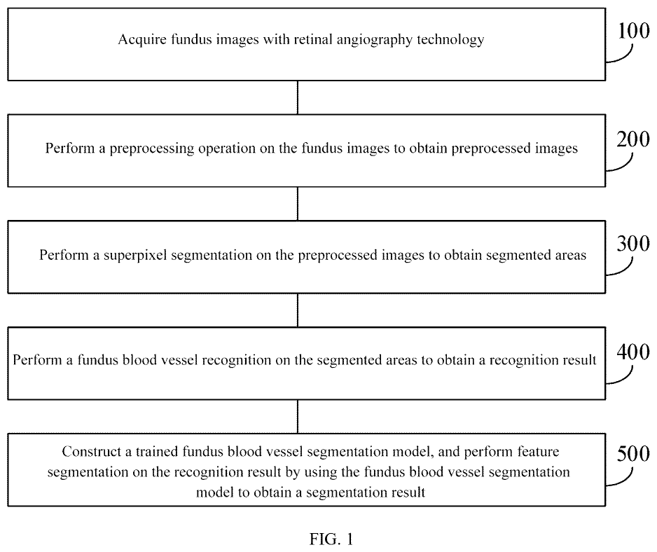

As shown in FIG. 1, the present disclosure provides a method for intelligent segmentation of fundus blood vessels based on retinal angiography, including the following steps:

Step 100: acquiring fundus images by a retinal angiography technology, where the fundus images include: a full field of view image and a local enhanced image;

Specifically, in the process of acquiring the fundus images using the retinal angiography (FFA) technology, the acquired images are recorded in real time to ensure that high-quality fundus retinal images are obtained, which contain distinct blood vessel structures, optic discs, and surrounding anatomical features. The full field of view image can cover most of the fundus area, while the local enhanced image focuses on capturing local areas with dense blood vessels. Each image needs to be taken at different time points in order to capture the dynamic changes and details of the fundus blood vessels.

More specifically, the size of the local enhanced image is usually between 512*512 pixels and 1024*1024 pixels, depending on the retinal imaging device used and its resolution. The size of the local enhanced image in this embodiment is 1024*1024 pixels. The number of blood vessels contained in a local enhanced image is generally between tens and hundreds, depending on the specific area analyzed and the complexity of the blood vessels. In some of the clearer, more blood vessel areas, approximately 100 to 300 blood vessels may be observed. In a smaller local area, especially when the focus is on major arteries and veins, the number may be between 20 and 50. In this embodiment, an image with more than 20 blood vessels is determined as a local enhanced image.

It should be noted that by recording the fundus images in real time, high-quality retinal images can be ensured, and the distinct blood vessel structure and anatomical features enable doctors to observe and analyze fundus conditions more clearly. The full field of view image can cover most of the fundus area, helping doctors to have a more comprehensive understanding of the overall condition of the fundus, while the local enhanced image can focus on local areas with dense blood vessels, making it easier to identify potential lesions or abnormalities. In addition, the high resolution (1024*1024 pixels) of the locally enhanced image makes subtle blood vessel changes and dynamic features more prominent, enhancing the ability to detect fundus lesions at an early stage. By comparing images taken at different time points, doctors can observe the dynamic changes in blood vessels, providing important basis for the progression of the disease and the evaluation of treatment effects. It can also effectively capture the number and complexity of blood vessels in a specific area, allowing doctors to make more accurate diagnoses based on the number and distribution of blood vessels during analysis, thereby improving the patient's treatment effect and providing strong technical support for the diagnosis and treatment of fundus diseases.

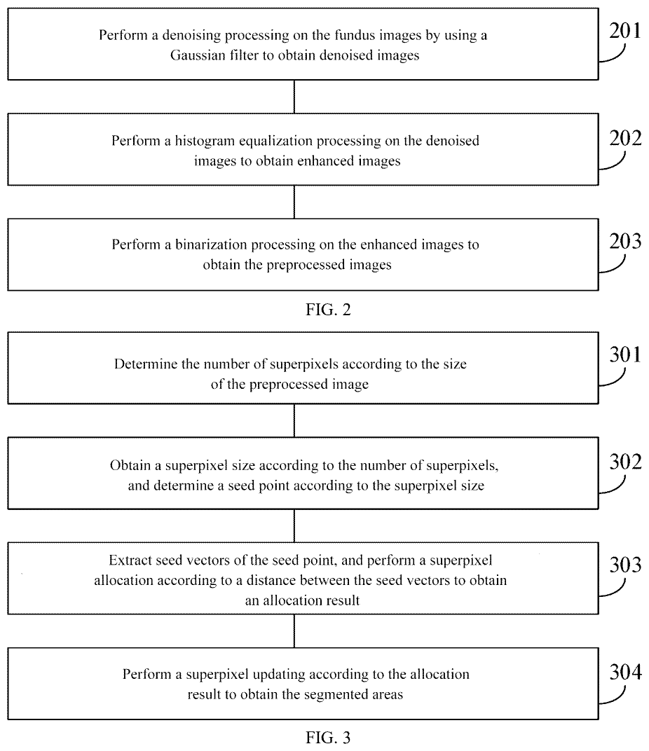

Step 200: performing a preprocessing operation on the fundus images to obtain preprocessed images; the specific steps are shown in FIG. 2, including:

Step 201: performing a denoising processing on the fundus images by using a Gaussian filter to obtain denoised images;

Specifically, filtering techniques (such as Gaussian filtering or median filtering) are used to reduce random noise in THE fundus images to improve image clarity. This embodiment uses the Gaussian filter to perform weighted averaging of the pixel values of each pixel and its surrounding neighborhood through a convolution operation, so that low-frequency information is retained and high-frequency noise in the image is suppressed, thereby generating a denoised image, which reduces the impact of background noise while ensuring structural features of the blood vessels.

Step 202: performing a histogram equalization processing on the denoised images to obtain enhanced images;

Specifically, histogram equalization redistributes the grayscale values of pixels in an image to make the brightness distribution in the image more uniform, especially between dark and bright areas. This improves the contrast of the image, making the difference between blood vessels and background more obvious and improving the clarity of visual information. It also ensures that details of smaller blood vessels are preserved, making the blood vessel features in the retina more prominent.

Step 203: performing a binarization processing on the enhanced images to obtain the preprocessed images.

Specifically, pixels are divided into foreground (usually blood vessels) and background parts using a fixed threshold or adaptive thresholding technique in order to highlight the blood vessel area. The optimal threshold in the processing process is determined by an Otsu algorithm.

It should be noted that Gaussian filtering effectively removes noise from the image and provides a clean basis for subsequent image analysis. Histogram equalization enhances the contrast of the image, making details and features more obvious, which can help doctors identify potential lesions more quickly. Through binarization processing, key structural features can be systematically extracted, making the subsequent analysis more focused and efficient. This series of preprocessing operations not only improves the clarity and readability of the image, but also provides important technical support for subsequent automated analysis, thereby improving the detection rate and diagnostic accuracy of fundus diseases.

Step 300: performing a superpixel segmentation on the preprocessed images to obtain segmented areas; the specific steps are shown in FIG. 3, including:

Step 301: determining the number K of superpixels according to the size of the preprocessed image;

Specifically, the number of superpixels K determines the segmentation accuracy of the image. If too many superpixels are specified, it will lead to insufficient detail features in some areas. The K value in this embodiment is 100.

Step 302: obtaining a superpixel size according to the number of superpixels, and determining a seed point according to the superpixel size;

Specifically, a calculation formula for the superpixel size is: S=\sqrt{N/K}; where: S is the expected size of each superpixel and N is the total number of pixels in the image. Then, the K seed points are uniformly selected on a certain segmented area of the preprocessed image, and the positions of the seed points are selected at intervals of S. These seed points will determine the center of the superpixel at the initial moment.

Step 303: extracting seed vectors of the seed point, and performing superpixel allocation according to a distance between the seed vectors to obtain an allocation result;

Specifically, the feature vector of each seed point (x, y) is calculated, including its spatial position and color information. The feature vector D is expressed as D=(x, y, L, a, b), where L, a, and b are the color values of the pixel in the CIE LAB color space. Then, the distance Dis from each pixel p in the image to each seed point cₖ is calculated based on the comprehensive measurement of spatial distance and color distance. The calculation formula is:

$$Dis = \sqrt{\begin{matrix}{( {x}_{p} - {x}_{{c}_{k}} )}^{2} + {( {y}_{p} - {y}_{{c}_{k}} )}^{2} + {( \frac{{L}_{p} - {L}_{{c}_{k}}}{{D}_{L}} )}^{2} + \\ {( \frac{{a}_{p} - {a}_{{c}_{k}}}{{D}_{a}} )}^{2} + {( \frac{{b}_{p} - {b}_{{c}_{k}}}{{D}_{b}} )}^{2}\end{matrix}} ;$$

where: (xₚ,yₚ) is the position of pixel p, (x_{c}_{ₖ},y_{c}_{ₖ}) is the position of seed point cₖ, Lₚ, aₚ, and bₚ are different color values of pixels in color space, L_{c}_{ₖ}, a_{c}_{ₖ}, and b_{c}_{ₖ} are different color values of seed points in color space, D_{L}, Dₐ, and D_{b} are color coordinate weights of different colors, which are used to balance the influence of different dimensions, D_{L}+Dₐ+D_{b}=1. Finally, according to the calculated distance, each pixel p is assigned to its nearest seed point.

Step 304: performing a superpixel updating according to the allocation result to obtain the segmented areas.

Specifically, the color values and positions of all pixels in each superpixel are counted, and a new seed point

$${c}_{k}^{n e w}$$

is calculated. The calculation formula for the new seed point is:

$${c}_{k}^{new} = ( \frac{1}{{N}_{k}} {\sum }_{p ∈ {c}_{k}} {x}_{p} , \frac{1}{{N}_{k}} {\sum }_{p ∈ {c}_{k}} {y}_{p} , \\ \frac{1}{{N}_{k}} {\sum }_{p ∈ {c}_{k}} {L}_{p} , \frac{1}{{N}_{k}} {\sum }_{p ∈ {c}_{k}} {a}_{p} , \frac{1}{{N}_{k}} {\sum }_{p ∈ {c}_{k}} {b}_{p} ) ;$$

where: Nₖ is the number of pixels belonging to cₖ. Step 303 is repeated until the center change of all superpixels is less than a predetermined threshold or the preset maximum number of iterations is reached. Finally, the superpixels of the new seed point are checked and the superpixels with too small areas are merged to obtain the segmented areas.

It should be noted that by specifying an appropriate number of superpixels K, the accuracy and effect of segmentation are ensured and the accuracy and detail expression of image segmentation are balanced. By evenly selecting seed points, it is ensured that different areas can be effectively segmented and represented, making the segmentation results more representative, thereby improving the stability and consistency of the results. By combining spatial features and color information to calculate the distance between each pixel and the seed point, the risk of misclassification due to a single feature parameter is reduced and the classification accuracy is improved. Through iterative updating and superpixel merging, the segmentation results can be continuously optimized, the accuracy of the segmented area can be enhanced, and the processing efficiency can be further improved, so that the final segmented area is more in line with the needs of medical image analysis.

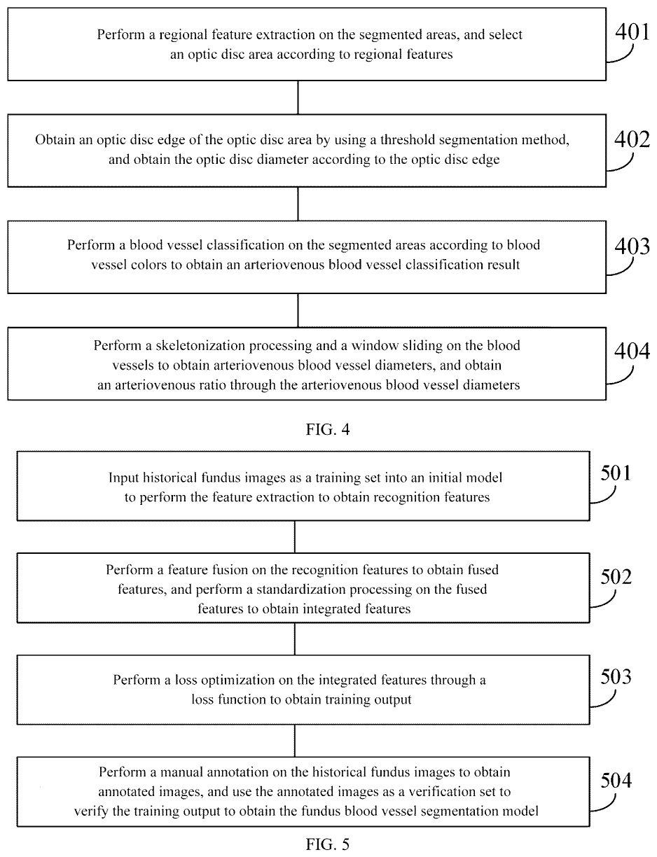

Step 400: performing a fundus blood vessel recognition on the segmented areas to obtain a recognition result, where the recognition result includes: an optic disc diameter, an arteriovenous blood vessel classification result, and an arteriovenous ratio; the specific steps are shown in FIG. 4, including:

Step 401: performing a regional feature extraction on the segmented areas, and selecting an optic disc area according to regional features;

Specifically, a regional growing or edge detection method is used to analyze and extract the boundary features and color distribution features of different areas. Based on the extracted features, possible optic disc areas are identified by a machine learning-based classifier. The recognition conditions include that the shape of the area has circular features and the color distribution features conform to the typical features of the optic disc (such as a brighter circle).

Step 402: obtaining an optic disc edge of the optic disc area by a threshold segmentation method, and obtaining the optic disc diameter according to the optic disc edge;

Specifically, a threshold is dynamically calculated according to the regional features by the Otsu method, and the optimal threshold is automatically determined by a grayscale histogram. The segmented area is divided into foreground (optic disc area) and background according to the optimal threshold. Then a Canny edge detection algorithm is used to calculate the gradient of the image to find the edge position. Finally, the images containing the edge positions are stitched together to obtain the optic disc edge.

More specifically, the edge contour of the optic disc is identified by a contour extraction algorithm, and the contour with the largest area is selected as the optic disc edge contour. The minimum circumscribed circle of the selected contour is calculated to obtain the center and radius information, and then all points on the contour are traversed to obtain the optic disc diameter by calculating the distance between the two farthest points covered by the contour.

Step 403: performing a blood vessel classification on the segmented areas according to blood vessel colors to obtain the arteriovenous blood vessel classification result;

Specifically, arterial and venous blood vessels are distinguished based on the color intensity, hue and diameter size of the blood vessels. Arteries are usually thinner and veins are thicker; the arteries are bright red and the veins are dark red, so the arteries are usually brighter in color than veins.

Step 404: performing a skeletonization processing and window sliding on the blood vessels to obtain arteriovenous blood vessel diameters, and obtaining the arteriovenous ratio through the arteriovenous blood vessel diameters.

Specifically, the classified arteriovenous blood vessels are skeletonized to extract the skeleton of the blood vessels, thereby simplifying the structure and retaining important shape information. Then a thinning algorithm is used to normalize the blood vessel width, and a skeleton line obtained after normalization will provide the blood vessel centerline. Then, a fixed window sliding is performed on the blood vessel skeleton through the window sliding technology, and the arterial diameter and venous diameter in the window are compared to obtain the arteriovenous ratio.

It should be noted that by combining regional feature extraction technology with threshold segmentation technology, the optic disc area and its edge can be effectively distinguished, ensuring accurate measurement of the optic disc diameter and providing a reliable basis for subsequent data analysis. Through blood vessel color analysis, accurate classification of arteriovenous blood vessels is achieved, providing doctors with intuitive indicators for evaluating blood vessel health status. The calculation of arteriovenous blood vessel diameter and arteriovenous ratio provides an important quantitative basis for monitoring the health status and pathological changes of fundus blood vessels, thereby improving the accuracy of image detection and enhancing the reliability of automated analysis.

Step 500: constructing a trained fundus blood vessel segmentation model, and performing feature segmentation on the recognition result by using the fundus blood vessel segmentation model to obtain a segmentation result, where a training process of the fundus blood vessel segmentation model includes: feature extraction, feature integration and loss function optimization.

Specifically, the training process of the fundus blood vessel segmentation model is shown in FIG. 5, including:

Step 501: inputting historical fundus images as a training set into an initial model to perform the feature extraction to obtain recognition features, where the recognition features include: color features, texture features, edge features, shape features, and intensity features.

Specifically, the initial model includes multi-scale convolution layers, hole convolution layers, and feature reconstruction layers connected in sequence. The multi-scale convolution layer is composed of multiple convolution layers. Multi-scale features are added to the first few layers of the model, and image features are processed in parallel through multiple convolution kernels of different sizes, thereby providing multi-level feature extraction from local to global. The hole convolution layer allows the model to have a more thorough understanding of the length and branches of blood vessels without increasing the number of parameters. The feature reconstruction layer is located at the end of the model. It reconstructs the blood vessel features through a deconvolution technology to generate high-definition images. Feature reconstruction utilizes upstream feature information and combines features at different levels with different weights to reconstruct the segmentation result.

More specifically, a large number of historical fundus images, including normal and abnormal fundus images, are collected to serve as a training set for the model. It is ensured that the training set is diverse, covering different patients, different disease conditions, and different imaging conditions. HSV color space is used to transform the historical fundus images to obtain the information of the three channels of hue, saturation and brightness. The color features are extracted using color histogram analysis and statistics such as the mean and variance in the color space. Threshold segmentation is performed to extract the blood vessel areas, and pixels within a specific range of hue values are determined and marked as potential blood vessel areas.

A Gabor filter and local binary patterns (LBP) are used to obtain texture features such as contrast, uniformity, and entropy of fundus images. The Gabor filter can effectively capture the subtle texture details of blood vessels by responding in different directions and scales. Using these features enables the model to better recognize the thickness, tortuosity, and branching features of blood vessels.

The Canny edge detection algorithm is used to extract edge features in fundus images and generate contour lines to enhance the boundary definition of the area. The algorithm first performs Gaussian filtering, then calculates the gradient and performs non-maximum suppression, and finally uses a double threshold method to determine the edge. Edge features help find the contours of the retina and blood vessels, and are crucial for the accurate positioning and classification of structures, especially providing clear boundary information for segmentation and subsequent analysis.

The centerline of the blood vessel is detected by the Hough transform technology, and the contour features (such as perimeter, area, shape factor, etc.) and shape descriptors are extracted. The Hough transform is used to detect straight lines and circles in images, and can extract the geometric shape information of blood vessels, further helping to establish a model of blood vessels and their distribution features. Shape features are used to analyze the geometric shapes of structures in fundus images. They can help identify the features of optic discs, blood vessels, and abnormal areas, and provide information that is helpful for disease diagnosis.

The grayscale mean, standard deviation, maximum and minimum grayscale values of the image are calculated to obtain the image brightness distribution, and the intensity changes in different areas are analyzed based on this. The intensity feature reflects the overall illumination and contrast conditions of the image, and can help supplement the color and texture features to further enhance the classification ability of the features.

Step 502: performing a feature fusion on the recognition features to obtain fused features, and performing a standardization processing on the fused features to obtain integrated features.

Further, the features extracted separately (color, texture, edge, shape and intensity features) are integrated to obtain fused features and expressed in the form of feature vector F, which is expressed as:

F=α(Y⊙W⊗B)+(β·Y+γ·X)⊗Q;
where: α is a weight coefficient calculated by a genetic algorithm, β and γ are adjustment coefficients obtained through training, which are used to dynamically adjust the influence of different features, β+γ=1, Y is a color feature, W is a texture feature, B is an edge feature, X is a shape feature, and Q is an intensity feature. The feature vectors are then processed using a Z-score normalization method to ensure that all features are within the same range.

Step 503: performing a loss optimization on the integrated features through a loss function to obtain training output.

Specifically, an expression of the loss function is:

$$L = - \frac{1}{N} {\sum }_{i = 1}^{N} ( ω \cdot {y}_{i} log ( {p}_{i} ) + ( 1 - ω ) \cdot ( 1 - {y}_{i} ) log ( 1 - {p}_{i} ) ) + λ \cdot ( 1 - \frac{2 \vert y ⋂ p \vert}{\vert y \vert + \vert p \vert} ) ;$$

where: L is the loss function, N is the total number of samples, y is a true label, p is a prediction probability, ω is a sample weight, λ is a coefficient weight, and i is the i-th pixel.

Step 504: performing a manual annotation on the historical fundus images to obtain annotated images, and using the annotated images as a verification set to verify the training output to obtain the fundus blood vessel segmentation model.

The present disclosure further provides a system for intelligent segmentation of fundus blood vessels based on retinal angiography, including:
an image acquisition module configured to acquire fundus images by a retinal angiography technology;
an image processing module configured to perform a preprocessing operation on the fundus images to obtain preprocessed images;
an image segmentation module configured to perform a superpixel segmentation on the preprocessed images to obtain segmented areas;
an image recognition module configured to perform a fundus blood vessel recognition on the segmented areas to obtain a recognition result; and
a model construction module configured to construct a trained fundus blood vessel segmentation model, and performing a feature segmentation on the recognition result by using the fundus blood vessel segmentation model to obtain a segmentation result.

The beneficial effects of the present disclosure are as follows.
1) A multi-stage image processing process is used, which includes image acquisition, preprocessing, superpixel segmentation, and blood vessel recognition. Each stage is optimized for a specific task, which improves the accuracy of the overall processing, reduces the need for manual intervention, and improves the efficiency of fundus image analysis;
2) Using a Gaussian filter noise reduction and a histogram equalization processing ensures that the image has higher contrast and clarity during segmentation and recognition, greatly reducing the interference of noise on subsequent analysis.
3) Through the superpixel algorithm, more accurate regional segmentation can be performed based on the image features and seed points, which can retain more detailed features and ensure the accuracy of subsequent further analysis of the blood vessels and optic disc areas.
4) By dynamically calculating the threshold through the Otsu method and combining with Canny edge detection, it is ensured that the optic disc edge and other key structures can be accurately extracted, thereby improving the accuracy of optic disc diameter and blood vessel classification.
5) Feature extraction includes color, texture, edge, shape and intensity features, which can fully reflect image information from multiple dimensions, provide sufficient feature information for model training, and enhance the learning and generalization capabilities of the model.
6) The initial model uses multi-scale convolution layers and hole convolution layers to process the image features in different ways, which can adapt to the complexity of various retinal images and ensure that the extracted blood vessel features are rich and accurate.

The various embodiments in this specification are described in a progressive manner, and each embodiment focuses on the differences from other embodiments. The same or similar parts between the various embodiments can be referenced to each other.

The present disclosure uses specific examples to illustrate the principles and implementations of the present disclosure. The descriptions of above embodiments are only used to help understand the method and core idea of the present disclosure. At the same time, for those of ordinary skill in the art, according to the concept of the present disclosure, there may be changes in the detailed description and application scopes. In summary, the contents of this specification should not be understood as limiting the present disclosure.

1. A method for intelligent segmentation of fundus blood vessels based on retinal angiography, comprising the following steps:
acquiring fundus images with retinal angiography technology, wherein the fundus images comprise: a full field of view image and a local enhanced image;
performing a preprocessing operation on the fundus images to obtain preprocessed images;
performing a superpixel segmentation on the preprocessed images to obtain segmented areas;
performing a fundus blood vessel recognition on the segmented areas to obtain a recognition result, wherein the recognition result comprises: an optic disc diameter, an arteriovenous blood vessel classification result, and an arteriovenous ratio; and
constructing a trained fundus blood vessel segmentation model, and performing a feature segmentation on the recognition result by using the fundus blood vessel segmentation model to obtain a segmentation result, wherein a training process of the fundus blood vessel segmentation model comprises: a feature extraction, a feature integration and a loss function optimization.

2. The method for intelligent segmentation of fundus blood vessels based on retinal angiography according to claim 1, wherein the full field of view image is an image covering an entire fundus range; the size of the local enhanced image is 1024*1024 pixels, and the number of blood vessels in the local enhanced image is greater than 20.

3. The method for intelligent segmentation of fundus blood vessels based on retinal angiography according to claim 1, wherein the performing the preprocessing operation on the fundus images to obtain the preprocessed images comprises:
performing a denoising processing on the fundus images by using a Gaussian filter to obtain denoised images;
performing a histogram equalization processing on the denoised images to obtain enhanced images; and
performing a binarization processing on the enhanced images to obtain the preprocessed images.

4. The method for intelligent segmentation of fundus blood vessels based on retinal angiography according to claim 1, wherein the performing the superpixel segmentation on the preprocessed images to obtain the segmented areas comprises:
determining the number of superpixels according to the size of the preprocessed image;
obtaining a superpixel size according to the number of superpixels, and determining a seed point according to the superpixel size;
extracting seed vectors of the seed point, and performing a superpixel allocation according to a distance between the seed vectors to obtain an allocation result; and
performing a superpixel updating according to the allocation result to obtain the segmented areas.

5. The method for intelligent segmentation of fundus blood vessels based on retinal angiography according to claim 4, wherein the distance between the seed vectors is calculated as follows:
$$Dis = \sqrt{\begin{matrix}{( {x}_{p} - {x}_{{c}_{k}} )}^{2} + {( {y}_{p} - {y}_{{c}_{k}} )}^{2} + {( \frac{{L}_{p} - {L}_{{c}_{k}}}{{D}_{L}} )}^{2} + \\ {( \frac{{a}_{p} - {a}_{{c}_{k}}}{{D}_{a}} )}^{2} + {( \frac{{b}_{p} - {b}_{{c}_{k}}}{{D}_{b}} )}^{2}\end{matrix}} ;$$
wherein: Dis is the vector distance, (xₚ,yₚ) is the position of pixel p, (x_{c}_{ₖ},y_{c}_{ₖ}) is the position of seed point cₖ, Lₚ, aₚ, and bₚ are different color values of the pixels in color space, L_{c}_{ₖ}, a_{c}_{ₖ}, and b_{c}_{ₖ} are different color values of the seed point in the color space, D_{L}, Dₐ, and D_{b} are color coordinate weights of different colors.

6. The method for intelligent segmentation of fundus blood vessels based on retinal angiography according to claim 1, wherein the performing the fundus blood vessel recognition on the segmented areas to obtain the recognition result comprises:
performing a regional feature extraction on the segmented areas, and selecting an optic disc area according to regional features;
obtaining an optic disc edge of the optic disc area by a threshold segmentation method, and obtaining the optic disc diameter according to the optic disc edge;
performing a blood vessel classification on the segmented areas according to blood vessel colors to obtain the arteriovenous blood vessel classification result; and
performing a skeletonization processing and a window sliding on the blood vessels to obtain arteriovenous blood vessel diameters, and obtaining the arteriovenous ratio through the arteriovenous blood vessel diameters.

7. The method for intelligent segmentation of fundus blood vessels based on retinal angiography according to claim 1, wherein the training process of the fundus blood vessel segmentation model comprises:
inputting historical fundus images as a training set into an initial model to perform the feature extraction to obtain recognition features, wherein the recognition features comprise: color features, texture features, edge features, shape features, and intensity features;
performing a feature fusion on the recognition features to obtain fused features, and performing a standardization processing on the fused features to obtain integrated features;
performing a loss optimization on the integrated features through a loss function to obtain training output; and
performing a manual annotation on the historical fundus images to obtain annotated images, and using the annotated images as a verification set to verify the training output to obtain the fundus blood vessel segmentation model.

8. The method for intelligent segmentation of fundus blood vessels based on retinal angiography according to claim 7, wherein the initial model comprises a multi-scale convolution layer, a hole convolution layer and a feature reconstruction layer connected in sequence; the multi-scale convolution layer is composed of multiple convolution layers, and the different convolution layers have built-in convolution kernels of different sizes; and the feature reconstruction layer has a built-in deconvolution algorithm.

9. The method for intelligent segmentation of fundus blood vessels based on retinal angiography according to claim 7, wherein an expression of the loss function is:
$$L = - \frac{1}{N} {\sum }_{i = 1}^{N} ( ω \cdot {y}_{i} log ( {p}_{i} ) + ( 1 - ω ) \cdot ( 1 - {y}_{i} ) log ( 1 - {p}_{i} ) ) + λ \cdot ( 1 - \frac{2 \vert y ⋂ p \vert}{\vert y \vert + \vert p \vert} ) ;$$
wherein L is the loss function, N is the total number of samples, y is a true label, p is a prediction probability, ω is a sample weight, λ is a coefficient weight, and i is the i-th pixel.

10. A system for intelligent segmentation of fundus blood vessels based on retinal angiography, comprising:
an image acquisition module configured to acquire fundus images by a retinal angiography technology;
an image processing module configured to perform a preprocessing operation on the fundus images to obtain preprocessed images;
an image segmentation module configured to perform superpixel segmentation on the preprocessed images to obtain segmented areas;
an image recognition module configured to perform fundus blood vessel recognition on the segmented areas to obtain a recognition result; and
a model construction module configured to construct a trained fundus blood vessel segmentation model, and performing feature segmentation on the recognition result by using the fundus blood vessel segmentation model to obtain a segmentation result.
